(19) United States
(12) Patent Application Publication
(10) Pub. No.: US 2010/0034746 A1
(43) Pub. Date: Feb. 11, 2010

(54) ANIMAL MODEL, SYSTEM, AND METHOD FOR SCREENING COMPOUNDS FOR ANTITHROMBOTIC AND/OR THROMBOLYTIC ACTIVITY

(75) Inventors: Ravindra Dattatraya Gupte, Mumbai (IN); Aditi Amol Tannu, Mumbai (IN); Radha Bhaskar Panicker, Mumbai (IN); Somesh Sharma, Mumbai (IN)

Correspondence Address: MERCHANT & GOULD PC, P.O. BOX 2903, MINNEAPOLIS, MN, 55402-0903, US

(73) Assignee: Piramal Life Sciences Limited, Mumbai (IN)

(21) Appl. No.: 12/532,009

(22) PCT Filed: Mar. 18, 2008
(86) PCT No.: PCT/IB08/51010

§ 371 Date: Sep. 18, 2009

Related U.S. Application Data
(60) Provisional application No. 60/920,445, filed on Mar. 27, 2007.

(51) Int. Cl. A61K 49/00; A61P 7/02

(52) U.S. Cl. 424/9.2

(57) ABSTRACT

The present invention relates to a method for evaluating a test compound for antithrombotic activity, thrombolytic activity, or a combination thereof. This method can include providing or employing a donor test animal and a recipient test animal. The donor and recipient test animals can have been pretreated with test compound. The donor test animal can be configured to provide oxygenated blood to the recipient test animal through a thrombus inducing system. This method also includes initiating transport of blood from the donor test animal to the recipient test animal through the thrombus inducing system. The method can include interrupting respiration of the recipient test animal and determining the length of time that the recipient test animal survives. In this method, a survival time longer than a predetermined threshold time indicates that the test compound has antithrombotic activity, thrombolytic activity, or a combination thereof.

### FIELD OF THE INVENTION

The present invention is directed to an animal model useful for screening compounds for antithrombotic and/or thrombolytic activity. The present invention relates to a method for evaluating a test compound for antithrombotic activity, thrombolytic activity, or a combination thereof. This method can include providing or employing a donor test animal and a recipient test animal. The donor and recipient test animals can have been pretreated with test compound. The donor test animal can be configured to provide oxygenated blood to the recipient test animal through a thrombus inducing system. This method also includes initiating transport of blood between the donor test animal and the recipient test animal through the thrombus inducing system. The method can include interrupting respiration of the recipient test animal and determining the length of time that the recipient test animal survives. In this method, a survival time longer than a predetermined threshold time indicates that the test compound has antithrombotic activity, thrombolytic activity, or a combination thereof.

### BACKGROUND OF THE INVENTION

Cardiovascular diseases, often the result of thrombotic events, is one of the leading causes of death worldwide.

Changes in the properties of blood constituents, obstruction of blood flow and internal injuries to the blood vessel wall, have been used for thrombogenesis in experimental animals.

Platelets, one of the blood constituents, play a critical role in hemostasis, as, when activated, they tend to aggregate and adhere to the site of injury thus initiating the clotting and the injury-sealing process. While efficient clotting at an external injury site limits the loss of blood, inappropriate formation of thrombi within the circulatory system due to an assault to vascular endothelium, obstructs normal blood flow and thus can result in life-threatening pathologies such as myocardial infarction, unstable angina, stroke, deep vein thrombosis, etc. Therefore, there exists a medical need to discover and develop efficacious antithrombotic and thrombolytic agents having minimal side effects, that can control and correct thrombotic disorders.

Animal models have always played a crucial role in drug discovery and development. Activities of many antithrombotic agents have been initially validated in various animal models of thrombosis and then successfully launched for the treatment and/or prevention of thrombotic disorders (Leadley et al., J. Pharmacol. Toxicol. Methods, (2000) 43:101-116). Examples are Activase®, (recombinant tissue plasminogen activator; Matsuo et al., Nature, (1981) 291:590-591), Abciximab (Coller et al., Blood, (1986) 68:783-786) and Hirudin (Agnelli et al., Thromb. Haemost., (1990) 63:204-207).

Some of the animal models are difficult to practice and require use of expensive instrumentation like laser beam apparatus or blood flow measuring device for measurement of cyclical flow changes. Some other models use very crude methods of assessment like measurement of the thrombus weight. This method may be inaccurate due to (a) the fragile nature of newly-formed thrombus, which disintegrates easily while being removed from the body and (b) contamination of the thrombus with blood and body fluids, which non-uniformly adds to its weight.

Since drug development is an expensive and time-intensive process, there exists a need for a simple, cost-effective, rapid and reproducible animal model to screen compounds for antithrombotic and/or thrombolytic activity.

### SUMMARY OF THE INVENTION

The present invention is directed to the development of an animal model useful for screening and identifying compounds for their antithrombotic and/or thrombolytic potential. The present invention relates to a method for evaluating a test compound for antithrombotic activity, thrombolytic activity, or a combination thereof. This method can include providing or employing a donor test animal and a recipient test animal. The donor and recipient test animals can have been pretreated with test compound. The donor test animal can be configured to provide oxygenated blood to the recipient test animal through a thrombus inducing system. This method also includes initiating transport of blood between the donor test animal and the recipient test animal through the thrombus inducing system. The method can include interrupting respiration of the recipient test animal and determining the length of time that the recipient test animal survives. In this method, a survival time longer than a predetermined threshold time indicates that the test compound has antithrombotic activity, thrombolytic activity, or a combination thereof.

In an aspect, the present invention is directed to a method to identify compounds useful for treating, reducing, or preventing thrombus formation in an experimental set-up using animals such as rats or guinea-pigs and that includes the following steps:
(a) in one experiment, connecting the cannula placed in the carotid artery of one anesthetized animal pretreated with vehicle to the cannula placed in the jugular vein of another anesthetized animal pretreated with vehicle and vice-versa by using a piece of latex tubing or damaged thoracic aorta and identifying one as a donor and another as a recipient;
(b) in another experiment, connecting the cannula placed in the carotid artery of one anesthetized animal pretreated with known antithrombotic agent to the cannula placed in the jugular vein of another anesthetized animal pretreated with the same antithrombotic agent and vice-versa by using a piece of latex tubing or damaged thoracic aorta and identifying one as a donor and another as a recipient;
(c) establishing cross circulation of blood between the animals by releasing the clamps placed on the vessels; and blocking trachea of recipient in both experiments (a) and (b);
(d) blocking of trachea as referred in (c) is achieved by tying it tightly with cotton thread after inserting a piece of polyethylene tubing in it, one end of which is heat sealed;
(e) survival time of the recipient in the experiments (a) and (b) is measured from the time of blocking its trachea.

### BRIEF DESCRIPTION OF THE DRAWINGS

FIG. 1 and FIG. 2 schematically illustrate an embodiment of the present thrombus inducing system as employed in the Examples 1 and 2 for carrying out an embodiment of the present method.

### DETAILED DESCRIPTION OF THE INVENTION

### Embodiments of the Present Method

The present invention relates to a method for evaluating a test compound for antithrombotic activity, thrombolytic activity, or a combination thereof. This method can include providing or employing a donor test animal and a recipient test animal. The donor and recipient test animals can have been pretreated with test compound. The donor test animal can be configured to provide oxygenated blood to the recipient test animal through a thrombus inducing system. This embodiment of the method also includes initiating transport of blood between the donor test animal and the recipient test animal through the thrombus inducing system. The method can include interrupting respiration of the recipient test animal and determining the length of time that the recipient test animal survives. In this method, a survival time longer than a predetermined threshold time indicates that the test compound has antithrombotic activity, thrombolytic activity, or a combination thereof.

### Test Animals

In an embodiment, the donor test animal and the recipient test animal are configured for cross-circulation between the donor test animal and the recipient test animal. Such cross-circulation can include an artery of the donor test animal in fluid communication with a vein of the recipient test animal and an artery of the recipient test animal in fluid communication with a vein of the donor test animal.

In an embodiment, the artery can be the carotid artery and the vein can be the jugular vein. An embodiment can employ a cannula in an artery of the donor test animal in fluid communication with the thrombus inducing system (e.g., a first thrombus inducing system) and the thrombus inducing system can be in fluid communication with a vein of the recipient test animal. An embodiment can employ a cannula in an artery of the recipient test animal in fluid communication with a vein of the donor test animal.

Fluid communication from the artery of the recipient test animal to the vein of the donor test animal can employ the thrombus inducing system (e.g., the first thrombus inducing system). For example, the cannula in the artery of the recipient test animal can be in fluid communication the thrombus inducing system (e.g., the first thrombus inducing system) and the thrombus inducing system can be in fluid communication with the vein of the donor test animal. In an embodiment, fluid communication from the artery of the recipient test animal to the vein of the donor test animal can employ a conduit that does not induce thrombus. The conduit can be, for example, tubing made of a material that does not induce thrombus (i.e., does not induce significant or adequate thrombus formation).

In an embodiment, the donor test animal can be configured to provide blood from a donor animal artery to a vein of the recipient animal. In an embodiment, the recipient test animal can be configured to provide blood from a recipient animal artery to a vein of the donor animal.

Initiating transport of blood can include opening a blocked conduit. For example, the blocked conduit can include an artery of the donor test animal. The method can include opening a plurality of blocked conduits. For example, the blocked conduit(s) can include an artery of the donor animal, a vein of the recipient animal, an artery of the recipient animal, a vein of the donor animal, or a combination thereof (i.e., more than one of the blocked conduits).

Interrupting respiration can include blocking the trachea of the recipient animal. For example, blocking the trachea can include inserting a sealed tube into the trachea and constricting the trachea around the sealed tube. In an embodiment, constricting the trachea around the sealed tube includes tying it tightly with cotton thread.

In an embodiment, initiating transport of blood and interrupting respiration are conducted over a period of about 5 to 30 seconds. For example, initiating transport of blood and interrupting respiration can be conducted concurrently over a period of about 10 to 15 seconds. By way of further example, initiating transport of blood and interrupting respiration can be conducted sequentially without operator initiated delay between initiating and interrupting. In an embodiment, initiating transport of blood and interrupting respiration are conducted sequentially without delay by the operator between initiating and interrupting.

In an embodiment, determining the length of time that the recipient test animal survives includes determining the elapsed time from interrupting respiration of the recipient test animal to death of the recipient test animal. Death of the recipient test animal can be considered to have happened at the time at which the animal ceases to make an effort to respire.

In an embodiment, the threshold time is the survival time of a control animal that was not pretreated or that was pretreated with a control substance lacking test compound and therapeutic agent or was sham treated.

The thrombus inducing system (e.g., the first, a second, or a third thrombus inducing system) can be or include any of a variety of known substances or apparatus effective to induce thrombus. For example, the thrombus inducing system can include latex, unsiliconised glass, stainless steel, freshly isolated piece of blood vessel, or combination thereof (i.e., more than one of these materials). For example, the thrombus inducing system can include latex tubing, unsiliconised glass capillary, stainless steel capillary, denuded abdominal aorta, or combination thereof (i.e., more than one of these conduits).

Known methods employing or evaluating biological materials or animals can employ replicates of the procedure, material, or animal. Results from a plurality of replicates of the procedure, material, or animal can be subjected to statistical analysis. In an embodiment, the present method includes providing a plurality of pairs of donor test animal and recipient test animal. For example, the method can include providing 10 pairs of animals each consisting of one donor and one recipient test animal.

The test compound evaluated by this method can be any of a variety of compounds, types of compounds, categories of compounds, mixtures of compounds, natural compounds (e.g., natural products or extracts including natural products), synthetic compounds, or the like. Methods for obtaining and handling such test compounds are known. Suitable test compounds include small molecules, herbal extracts, microbial extracts, drugs, antibodies, peptides, or secreted proteins. The term “small molecules” means molecules having a molecular weight up to 1200.

The test, control, or treated animal can be any of a variety of types of animals commonly used in laboratories for pharmacological or toxicological testing. The animal is a non-human animal. In an embodiment, the animal is a non-primate animal. In an embodiment, the animal is a rodent. Suitable animals include rat, guinea-pig, mouse, hamster, or rabbit. In an embodiment the animal is a rat.

### Providing Test Animals

In certain embodiments the present method can include providing a pair of animals. The method can also include treating each of the animals with test compound. Such an embodiment can include designating one of the animals as the donor test animal and the other animal as the recipient test animal. In addition, the method can include configuring the donor test animal to provide oxygenated blood to the recipient test animal through a thrombus inducing system (e.g., a first thrombus inducing system).

In an embodiment, configuring can include establishing cross-circulation between the donor test animal and the recipient test animal. Such establishing can include putting an artery of the donor test animal into fluid communication with a vein of the recipient test animal and putting an artery of the recipient test animal into fluid communication with a vein of the donor test animal. In an embodiment, the artery is the carotid artery and the vein is the jugular vein.

The method can employ any of a variety of methods for establishing fluid communication or putting one vessel in fluid communication with another vessel. For example, the method can include placing a cannula in an artery of the donor test animal. The cannula can be in fluid communication with the thrombus inducing system (e.g., the first thrombus inducing system) and the thrombus inducing system can be in fluid communication with the vein of the recipient test animal. In an embodiment, the method includes placing a cannula in an artery of the recipient test animal. The cannula can be in fluid communication with the thrombus inducing system (e.g., the first thrombus inducing system) and the thrombus inducing system can be in fluid communication with the vein of the donor test animal. In an embodiment, the cannula can be in fluid communication with the vein of the donor control animal through a conduit that does not induce thrombus. The conduit can be, for example, tubing made of a material that does not induce thrombus (i.e., does not induce significant or adequate thrombus formation).

In an embodiment, the method can include configuring the donor test animal to provide blood from a donor animal artery to a vein of the recipient animal. The method can also include configuring the recipient test animal to provide blood from a recipient animal artery to a vein of the donor test animal.

In an embodiment, the present method includes treating a plurality of pairs of donor test animal and recipient test animal. For example, the method can include treating 10 pairs of animals each consisting of one donor and one recipient test animal.

### Control Animals

Known methods employing or evaluating biological materials or animals can employ a control procedure, material, or animal. Results from the control procedure, material, or animal can be employed, for example, to provide a control value to which a test result is compared to determine its significance. For example, it may be desirable for a test compound to induce survival longer than survival of a control group.

In an embodiment, the present method includes a control procedure. This control procedure can include, for example, providing a donor control animal and a recipient control animal. The donor and recipient control animals can each have been pretreated with control substance. The donor control animal can be configured to provide oxygenated blood to the recipient control animal through a thrombus inducing system (e.g., a second thrombus inducing system). This embodiment can also include initiating transport of blood from the donor control animal to the recipient control animal through the thrombus inducing system (e.g., the second thrombus inducing system). The method can include interrupting respiration of the recipient control animal and determining the length of time that the recipient control animal survives.

In an embodiment, the donor control animal and the recipient control animal can be configured for cross-circulation between the donor control animal and the recipient control animal. For example, an artery of the donor control animal can be in fluid communication with a vein of the recipient control animal and an artery of the recipient control animal can be in fluid communication with a vein of the donor control animal.

In an embodiment, the artery is the carotid artery and the vein is the jugular vein. For example, a cannula in an artery of the donor control animal can be in fluid communication with the thrombus inducing system (e.g., the second thrombus inducing system) and the thrombus inducing system can be in fluid communication with the vein of the recipient control animal. In an embodiment, a cannula in an artery of the recipient control animal is in fluid communication with a vein of the donor control animal.

Fluid communication from the artery of the recipient control animal to the vein of the donor control animal can employ the thrombus inducing system (e.g., the second thrombus inducing system). For example, the cannula in the artery of the recipient control animal can be in fluid communication the thrombus inducing system (e.g., the second thrombus inducing system) and the thrombus inducing system can be in fluid communication with the vein of the donor control animal. In an embodiment, fluid communication from the artery of the recipient control animal to the vein of the donor control animal can employ a conduit that does not induce thrombus. The conduit can be, for example, tubing made of a material that does not induce thrombus (i.e., significant or adequate thrombus formation).

In an embodiment, the donor control animal is configured to provide blood from a donor animal artery to a vein of the recipient animal. In an embodiment, the recipient control animal is configured to provide blood from a recipient animal artery to a vein of the donor animal.

In an embodiment, initiating transport of blood includes opening a blocked conduit. For example, the blocked conduit can be or include an artery of the donor control animal. In an embodiment, the method can include opening a plurality of blocked conduits. The blocked conduit(s) can include an artery of the donor control animal, a vein of the recipient control animal, an artery of the recipient control animal, a vein of the donor control animal, or a combination thereof (i.e., more than one of the blocked conduits).

In an embodiment, interrupting respiration includes blocking the trachea of the recipient control animal. For example, blocking the trachea can include inserting a sealed tube into the trachea and constricting the trachea around the sealed tube. In an embodiment, constricting the trachea around the sealed tube can include tying it tightly with cotton thread.

In an embodiment, determining the length of time that the recipient control animal survives includes determining the elapsed time from interrupting respiration of the recipient control animal to death of the recipient control animal. For example, the death of the recipient control animal can be considered to be the time at which the animal ceases to make an effort to respire.

In an embodiment, initiating transport of blood and interrupting respiration are conducted over a period of about 5 to 30 seconds. For example, initiating transport of blood and interrupting respiration are conducted concurrently over a period of 10 to 15 seconds. By way of further example, initiating transport of blood and interrupting respiration are conducted sequentially without operator initiated delay between initiating and interrupting. In an embodiment, initiating transport of blood and interrupting respiration are conducted sequentially without delay by the operator between initiating and interrupting.

Known methods employing or evaluating biological materials or animals can employ replicates of the control procedure, material, or animal. Results from a plurality of replicate controls can be subjected to statistical analysis. In an embodiment, the present method includes providing a plurality of pairs of donor control animal and recipient control animal. For example, the method can include providing 10 pairs of animals each consisting of one donor and one recipient control animal.

Known methods employing or evaluating test compounds in biological materials or animals employ any of a variety of controls for the test compound. In an embodiment, the present method employs a control substance that has been administered to the control animals. The control substance can be any of a variety of known control substances or categories of control substances. In certain embodiments, the control substance can be or include vehicle. In certain embodiments, the control substance can be or include an inactive compound that does not affect platelets, such as metformin. A compound such as metformin, which does not affect platelets, may be used as a negative control at a dose of 300 mg/kg, p.o. In an embodiment, the control animal has been subjected to sham treatment. For example, the donor and recipient control animals have been pretreated with control substance or have been sham treated.

In an embodiment, the length of time that the recipient control animal survives is the predetermined threshold time. In an embodiment, the predetermined threshold time equals the length of time that the recipient control animal survives multiplied by a predetermined number greater than 1.

### Providing Control Animals

In certain embodiments the present method can include providing a pair of animals. The method can also include treating each of the animals with control substance or sham treating the animals. Such an embodiment can include designating one of the animals as the donor control animal and the other animal as the recipient control animal. In addition, the method can include configuring the donor control animal to provide oxygenated blood to the recipient control animal through a thrombus inducing system (e.g., the second thrombus inducing system).

In an embodiment, configuring can include establishing cross-circulation between the donor control animal and the recipient control animal. Such establishing can include putting an artery of the donor control animal into fluid communication with a vein of the recipient control animal and putting an artery of the recipient control animal into fluid communication with a vein of the donor control animal. In an embodiment, the artery is the carotid artery and the vein is the jugular vein.

The method can employ any of a variety of methods for establishing fluid communication or putting one vessel in fluid communication with another vessel. For example, the method can include placing a cannula in an artery of the donor control animal. The cannula can be in fluid communication with the thrombus (e.g., a second thrombus inducing system) inducing system and the thrombus inducing system can be in fluid communication with the vein of the recipient control animal. In an embodiment, the method includes placing a cannula in an artery of the recipient control animal. This cannula can be in fluid communication with the thrombus inducing system (e.g., a second thrombus inducing system) and the thrombus inducing system can be in fluid communication with the vein of the donor control animal. In an embodiment, the cannula can be in fluid communication with the vein of the donor control animal through a conduit that does not induce thrombus. The conduit can be, for example, tubing made of a material that does not induce thrombus (i.e., does not induce significant or adequate thrombus formation).

In an embodiment, the method includes configuring the donor control animal to provide blood from a donor animal artery to a vein of the recipient animal. The method can also include configuring the recipient control animal to provide blood from a recipient animal artery to a vein of the donor animal.

In an embodiment, the present method includes treating a plurality of pairs of donor control animal and recipient control animal. For example, the method can include treating 10 pairs of animals each consisting of one donor and one recipient control animal.

### Treated Animals

Known methods employing or evaluating biological materials or animals can employ a positive control, that is, a biological material or animal that has been treated with a known therapeutic agent or active compound. Results from the animals treated with a known therapeutic agent or active compound can provide a result or value from a procedure to which a test result is compared to determine its significance. For example, it may be desirable for a test compound to induce survival as long as or longer than the survival of animals treated with therapeutic agent or active compound.

In an embodiment, the present method includes employing an animal that has been treated with a known antithrombotic agent, thrombolytic agent, or combination or mixture thereof. This treatment procedure can include, for example, providing a donor treated animal and a recipient treated animal. The donor and recipient treated animals can each have been pretreated with antithrombotic agent, thrombolytic agent, or combination or mixture thereof. The donor treated animal can be configured to provide oxygenated blood to the recipient treated animal through a thrombus inducing system (e.g., a third thrombus inducing system). This embodiment can also include initiating transport of blood from the donor treated animal to the recipient treated animal through the thrombus inducing system (e.g., the third thrombus inducing system). The method can include interrupting respiration of the recipient treated animal and determining the length of time that the recipient treated animal survives.

In an embodiment, the donor treated animal and the recipient treated animal can be configured for cross-circulation between the donor treated animal and the recipient treated animal. For example, an artery of the donor treated animal can be in fluid communication with a vein of the recipient treated animal and an artery of the recipient treated animal can be in fluid communication with a vein of the donor treated animal.

In an embodiment, the artery is the carotid artery and the vein is the jugular vein. For example, a cannula in an artery of the donor treated animal can be in fluid communication with the thrombus inducing system (e.g., a third thrombus inducing system) and the thrombus inducing system can be in fluid communication with the vein of the recipient treated animal. In an embodiment, a cannula in an artery of the recipient treated animal is in fluid communication with the vein of the donor treated animal.

Fluid communication from the artery of the recipient treated animal to the vein of the donor treated animal can employ the thrombus inducing system (e.g., the third thrombus inducing system). For example, the cannula in the artery of the recipient treated animal can be in fluid communication the thrombus inducing system (e.g., the third thrombus inducing system) and the thrombus inducing system can be in fluid communication with the vein of the donor treated animal. In an embodiment, fluid communication from the artery of the recipient treated animal to the vein of the donor treated animal can employ a conduit that does not induce thrombus. The conduit can be, for example, tubing made of a material that does not induce thrombus (i.e., does not induce significant or adequate thrombus formation).

In an embodiment, the donor treated animal is configured to provide blood from a donor animal artery to a vein of the recipient animal. In an embodiment, the recipient treated animal is configured to provide blood from a recipient animal artery to a vein of the donor animal.

In an embodiment, initiating transport of blood includes opening a blocked conduit. For example, the blocked conduit can be or include an artery of the donor treated animal. In an embodiment, the method can include opening a plurality of blocked conduits. The blocked conduit(s) can include an artery of the donor treated animal, a vein of the recipient treated animal, an artery of the recipient treated animal, a vein of the donor treated animal, or a combination thereof (i.e., more than one of the blocked conduits).

In an embodiment, interrupting respiration includes blocking the trachea of the recipient treated animal. For example, blocking the trachea can include inserting a sealed tube into the trachea and constricting the trachea around the sealed tube. In an embodiment, constricting the trachea around the sealed tube can include tying it tightly with cotton thread.

In an embodiment, determining the length of time that the recipient treated animal survives includes determining the elapsed time from interrupting respiration of the recipient treated animal to death of the recipient treated animal. For example, the death of the recipient treated animal can be considered to be the time at which the animal ceases to make an effort to respire.

In an embodiment, initiating transport of blood and interrupting respiration are conducted over a period of about 5 to 30 seconds. For example, initiating transport of blood and interrupting respiration are conducted concurrently over a period of about 10 to 15 seconds. By way of further example, initiating transport of blood and interrupting respiration are conducted sequentially without operator initiated delay between initiating and interrupting. In an embodiment, initiating transport of blood and interrupting respiration are conducted sequentially without delay by the operator between initiating and interrupting.

Known methods employing or evaluating biological materials or animals can employ replicates of the treatment procedure, material, or animal. Results from a plurality of replicate treatments can be subjected to statistical analysis. In an embodiment, the present method includes providing a plurality of pairs of donor treated animal and recipient treated animal. For example, the method can include providing 10 pairs of animals each consisting of one donor and one recipient treated animal.

The present method can include providing animals pretreated with antithrombotic agent. The present method can include providing animals pretreated with thrombolytic agent.

In an embodiment, the length of time that the recipient treated animal survives is the predetermined treatment survival time.

The present method can employ any of a variety of known antithrombotic agents, thrombolytic agents, or combination or mixture thereof. Suitable antithrombotic agents or thrombolytic agents include acetylsalicylic acid (Aspirin), dipyridamole, clopidogrel bisulphate, heparin, GPIIb/IIIa receptor inhibitors (e.g., ReoPro, Aggrastat, Integrilin), platelet aggregation inhibitor (e.g., Plavix), tissue plasminogen activator (e.g., Activase, Retavase), streptokinase, urokinase, another thrombus lysing agent, or combination or mixture thereof.

Suitable antithrombotic agents include acetylsalicylic acid (Aspirin), dipyridamole, clopidogrel bisulphate, warfarin, heparin (e.g. Lovenox), GPIIb/IIIa receptor inhibitors (e.g. ReoPro, Aggrastat, Integrilin), ticlopidine, sulfinpyrazone, or combination or mixture thereof. Preferred antithrombotic agents include acetylsalicylic acid (Aspirin), dipyridamole, clopidogrel bisulphate, or combination or mixture thereof. Suitable doses for these antithrombotic agents are known. For example, known agents such as acetylsalicylic acid (Aspirin), dipyridamole, clopidogrel bisulphate can be used at their effective doses selected from 15 to 25 mg/kg p.o.

Suitable thrombolytic agents include tissue plasminogen activator (e.g., Activase, Retavase), streptokinase, urokinase, or combination or mixture thereof. Preferred thrombolytic agents include streptokinase. Suitable doses for these thrombolytic agents are known. For example, streptokinase can be used at a dose of 50000-70000 I.U./ml/kg/hour intravenously, as a continuous infusion.

### Providing Treated Animals

In certain embodiments the present method can include providing a pair of animals. The method can also include treating each of the animals with antithrombotic agent, thrombolytic agent, or combination or mixture thereof. Such an embodiment can include designating one of the animals as the donor treated animal and the other animal as the recipient treated animal. In addition, the method can include configuring the donor treated animal to provide oxygenated blood to the recipient treated animal through a thrombus inducing system (e.g., a third thrombus inducing system).

In an embodiment, configuring can include establishing cross-circulation between the donor treated animal and the recipient treated animal. Such establishing can include putting an artery of the donor treated animal into fluid communication with a vein of the recipient treated animal and putting an artery of the recipient treated animal into fluid communication with a vein of the donor treated animal. In an embodiment, the artery is the carotid artery and the vein is the jugular vein.

The method can employ any of a variety of methods for establishing fluid communication or putting one vessel in fluid communication with another vessel. For example, the method can include placing a cannula in an artery of the donor treated animal. The cannula can be in fluid communication with the thrombus inducing system (e.g., the third thrombus inducing system) and the thrombus inducing system can be in fluid communication with the vein of the recipient treated animal. In an embodiment, the method includes placing a cannula in an artery of the recipient treated animal. This cannula can be in fluid communication with the thrombus inducing system (e.g., the third thrombus inducing system) and the thrombus inducing system can be in fluid communication with the vein of the donor treated animal. In an embodiment, the cannula can be in fluid communication with the vein of the donor treated animal through a conduit that does not induce thrombus. The conduit can be, for example, tubing made of a material that does not induce thrombus (i.e., does not induce significant or adequate thrombus formation).

The method can also include configuring the donor treated animal to provide blood from a donor animal artery to a vein of the recipient treated animal. The method can also include configuring the recipient treated animal to provide blood from a recipient animal artery to a vein of the donor treated animal.

In an embodiment, the present method includes treating a plurality of pairs of donor treated animal and recipient treated animal. For example, the method can include treating 10 pairs of animals each consisting of one donor and one recipient treated animal.

### Additional Embodiments of the Present Invention

The present invention is also directed to the experimental set-up using small laboratory animals, to study antithrombotic or thrombolytic potential of compounds. Particularly, the present invention relates to the induction of thrombus formation in an experimental set-up using small laboratory animals such as rats or guinea pigs, by connecting the cannulated blood vessels of experimental animals using a connector such as piece of latex tubing, unsiliconised glass capillary, stainless steel capillary or even a freshly isolated blood vessel piece like denuded abdominal aorta, from an animal specially sacrificed for the purpose. Thus, the present invention permits the study of compounds for their possible use as antithrombotic or thrombolytic agents.

In one aspect, the present invention is directed to the method of identifying compounds useful for treating, reducing, or preventing thrombus formation in an experimental set-up using small laboratory animals. An embodiment of the method can include, for example:
(a) in one experiment, connecting the cannula placed in the carotid artery of one anesthetized animal pretreated with vehicle to the cannula placed in the jugular vein of another anesthetized animal pretreated with vehicle and vice-versa by using a piece of latex tubing or damaged thoracic aorta and identifying one as a donor and another as a recipient;
(b) in another experiment, connecting the cannula placed in the carotid artery of one anesthetized animal pretreated with known antithrombotic agent to the cannula placed in the jugular vein of another anesthetized animal pretreated with the same antithrombotic agent and vice-versa by using a piece of latex tubing or damaged thoracic aorta and identifying one as a donor and another as a recipient;
(c) establishing cross circulation of blood between the animals by releasing the clamps placed on the vessels; and blocking trachea of recipient in both experiments (a) and (b);
(d) blocking of trachea as referred in (c) is achieved by tying it tightly with cotton thread after inserting a piece of polyethylene tubing in it, one end of which is heat sealed;
(e) survival time of a recipient in the experiments (a) and (b) is measured from the time of blocking it's trachea.

An antithrombotic agent or thrombolytic agent identified by the method of the present invention can be utilized to treat disease states associated with thrombosis. As used herein, “thrombosis” is the process of intravascular formation of thrombi including fibrin and platelets that cause hindrance to normal blood flow. Disease states associated with thrombosis include, but are not limited to, myocardial infarction, atherosclerosis, restenosis after angioplasty or coronary artery bypass graft, stroke, coronary artery disease, deep vein thrombosis, unstable angina, etc.

The present invention may be better understood with reference to the following examples. These examples are intended to be representative of specific embodiments of the invention, and are not intended as limiting the scope of the invention.

### Example 1

### Step 1

### Procedure

Wistar rats (either sex; weighing 290-310 g) were used throughout the experiments. Animals were housed and cared for in accordance with the Guidelines in force published by CPCSEA (Committee for the Purpose of Control and Supervision of Experiments on Animals), Tamil Nadu, India. Procedures using laboratory animals were approved by the IAEC (Institutional Animal Ethics Committee) of the Research Centre of Nicholas Piramal India Limited, Mumbai, India.

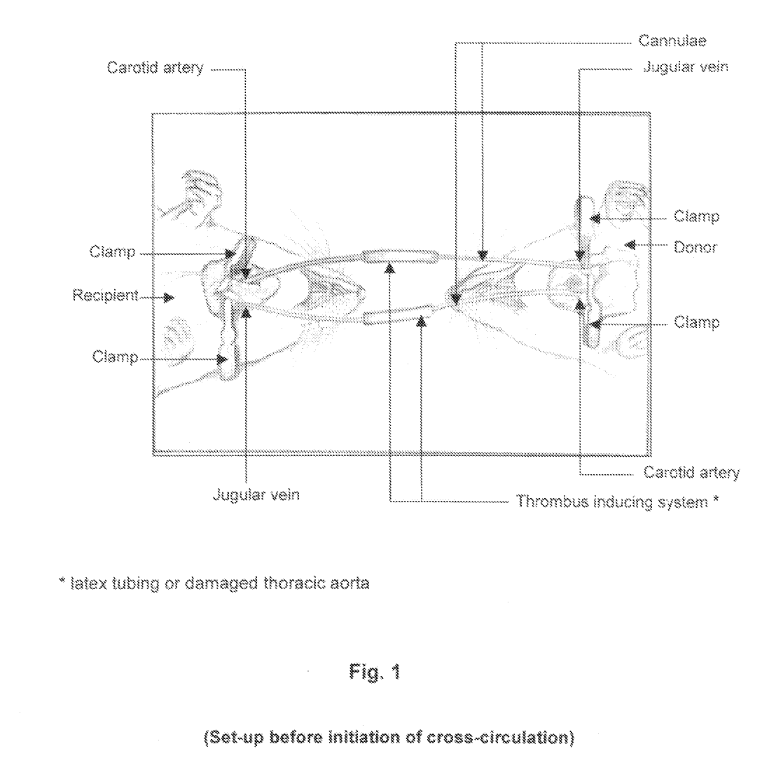

In one experiment a pair of rats were anesthetized using urethane (1.5 g/kg; Fluka) by intra-peritoneal administration. After anesthetization, one of the jugular veins and carotid arteries of each of the rats were exposed and clamped. Polyethylene cannulae (0.76 mm ID and 1.22 mm OD, polyethylene tubing, each 6.0 cm in length; Portex Ltd) filled with normal saline (0.9%, w/v, sodium chloride, Merck) were introduced into these vessels and secured tightly by tying with cotton thread (No. 40, Madura Coats Ltd., India). Furthermore, trachea of any one of the rats was exposed and a piece of thread was put loosely around it (recipient). Then the arterial cannula of one of the rats was connected to the venous cannula of the other rat and vice versa, using 2.5 cm long piece of latex tubing (0.89 mm ID; PharMed, Cole-Parmer). FIG. 1 schematically illustrates these rats.

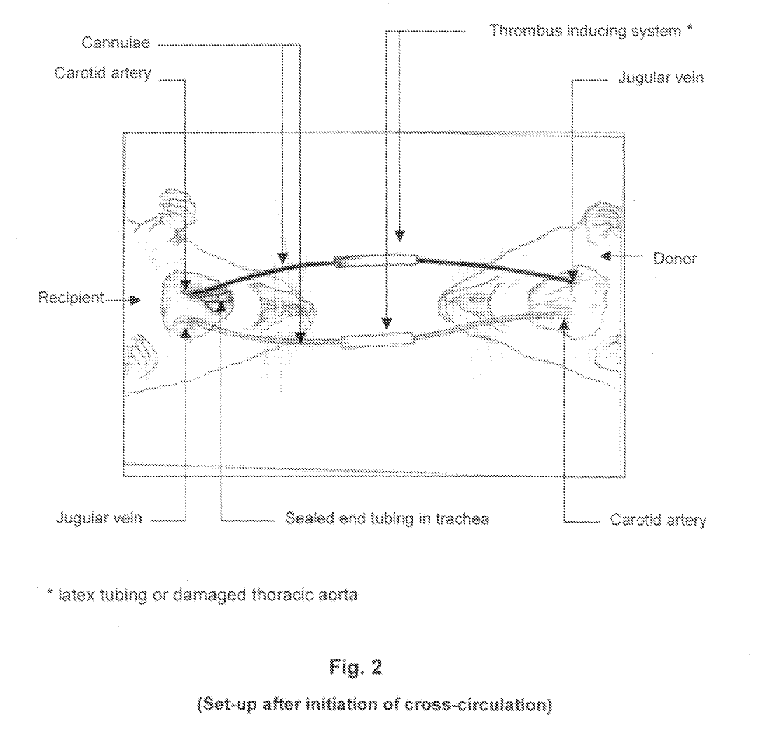

The experiment was initiated by opening the clamps on all the vessels, which initiated cross circulation of blood between the two animals. Then the trachea of the recipient was completely blocked by inserting a piece of polyethylene tubing (1.7 mm ID and 2.7 mm OD; Portex Ltd, one end of which was heat-sealed) into it, and subsequently tying it tightly with cotton thread. FIG. 2 schematically illustrates these rats.

In spite of the blocked trachea, the recipient survived, as long as the oxygenated blood supply from the donor continued. Survival was monitored by the animal's effort to respire. During the process of cross circulation, when blood passed through the cannula, the platelets came in contact with the internal surface of the latex tubing and thus got activated. This process led to thrombogenesis, blocked the cannulae, thereby cutting off the supply of the oxygenated blood to the recipient, thus leading to its death.

### Step 2

### Drug administration

In another experiment, pairs of rats were treated with antithrombotic/thrombolytic agents selected from the following list:

(a) Aspirin, 20.0 mg/kg, p.o.

(b) Dipyridamole, 20.0 mg/kg, p.o.

(c) Clopidogrel bisulphate, 20.0 mg/kg, p.o.

(d) Metformin, 300.0 mg/kg, p.o. (negative control)

(e) Streptokinase, 60,000 I.U./ml/kg/hour, i.v., as continuous infusion

The drug suspensions for oral use were prepared in the vehicle, carboxymethyl cellulose (CMC, 0.5% w/v, Sigma), using polyoxyethylenesorbitan monooleate (Tween 80, Sigma) as a wetting agent. Commercially available streptokinase solution was diluted with normal saline to an appropriate concentration for the i.v. use.

Vehicle/drugs were administered to rats orally at the doses mentioned above, in a volume of 1 ml/kg. With the exception of streptokinase, all other drugs were administered once daily for three consecutive days and the experiment was performed on the third day, one hour after the administration of the last dose. The vehicle-treated animals were used as controls.

Streptokinase was administered as a continuous infusion at a dose of 60,000 I.U./ml/kg/hour intravenously, through the femoral vein of the recipient and was administered only on the day of experiment. As a source of plasminogen, human plasma was injected to the animal intravenously at a dose of 1 ml/kg, just before blocking the trachea. Streptokinase infusion was initiated two minutes after blocking the trachea of the recipient. Saline-infused animals were used as control for streptokinase-treated animals.

The survival time of the recipients in the abovementioned experiments was recorded. The results are presented in Table 1.

TABLE 1

| | | | | | | Survival time |
|---|---|---|---|---|---|---|
| Sr. | | | | | No. of | (min) |
| No. | Treatment | Route | Dose | | observations* | Mean ± S.E. |
| 1 | CMC (control) | p.o. | 1 | ml/kg. | 20 | 10.9 ± 0.70 |
| 2 | Aspirin | p.o. | 20 | mg/kg | 11 | 45.3 ± 4.40*** |
| 3 | Dipyridamole | p.o. | 20 | mg/kg | 10 | 38.5 ± 1.55*** |
| 4 | Clopidogrel | p.o. | 20 | mg/kg | 10 | 47.8 ± 3.78*** |
| 5 | Metformin | p.o. | 300 | mg/kg | 10 | 7.9 ± 1.75 n.s. |
| 6 | Saline | i.v. | 1 | ml/kg/hr | 10 | 10.4 ± 0.91 |
| | (control) | infusion | | | | |
| 7 | Streptokinase | i.v. | 60,000 | IU/ml/kg/hr | 10 | 89.5 ± 10.55*** |
| | | infusion | | | | |

*= For each observation one pair of animals consisting of one donor and one recipient animal were used
S.E. = standard error;
***= p < 0.001
n.s. = not significant, when compared with corresponding control groups.

### Conclusion:

In the present invention, the creation of a novel model of thrombosis in an experimental set-up using rats has been described. It is clear from the results of the experiments that the process of thrombus formation was delayed in the animals which were pretreated with known antithrombotic/thrombolytic agents as compared with vehicle treated animals, and hence a significant prolongation of the survival time of the recipients occurred. A significant prolongation of the survival time of the recipients also occurred with streptokinase treatment. The results clearly indicate that the method can be used for screening and identifying compounds with antithrombotic and thrombolytic potential.

### Example 2

### Rationale

In the present experiment, the piece of latex tubing used in Example 1 was replaced by a piece of rat thoracic aorta with damaged endothelium, so that the anti-thrombotic effect of the compounds could be studied in a near natural situation.

Maintenance of a patent vasculature is critical to provide blood flow to dependent tissues. This is normally facilitated by vessels composed of actively non-thrombogenic endothelium and blood that contains both non-activated platelets and inactive coagulation proenzymes. Following vessel injury, active hemostasis results from vasoconstriction, adherence of activated platelets to damaged endothelium, their aggregation and activation of coagulation enzymes, finally resulting into thrombosis. It has been shown that chronic carbon tetrachloride treatment to rats causes serious damage to vascular endothelial cells and thus the blood vessels get damaged. It was thought of using these damaged vessels, instead of latex tubing (used in Example 1), to connect arterial and venous cannulae of donor and recipient, in order to mimic the physiological process of thrombus formation.

Step 1: Preparation of Rat Thoracic Aorta with Damaged Endothelium

The procedure used is generally as described in the reference, Physiol. Res., (2006) 55: 245-251, the disclosure of which is incorporated by reference for the teaching of the experiment.

Wistar rats (male, weighing 290-310 g) were used throughout the experiments. Animals were housed and cared for, in accordance with the Guidelines in force published by CPCSEA (Committee for the Purpose of Control and Supervision of Experiments on Animals), Tamil Nadu, India. Procedures using laboratory animals were approved by the IAEC (Institutional Animal Ethics Committee) of the Research Center of Nicholas Piramal India Limited, Mumbai, India.

They were administered carbon tetrachloride (Fluka) in olive oil (Figaro, Spain) (1:1 v/v), 0.5 ml/kg, intraperitoneally, twice a week for eight continuous weeks. The body weight range of the animals at this stage was 360-390 g. Four days after the last administration of carbon tetrachloride, complete thoracic aortae of these animals were removed and used immediately for the experiments mentioned below.

### Step 2: Procedure

Wistar rats (either sex; weighing 290-310 g) were used for the experiments.

In one experiment a pair of rats was anesthetized with urethane (1.5 g/kg; Fluka) by intra-peritoneal administration. After anesthetization, one of the jugular veins and carotid arteries of each of the rats were exposed and clamped. Polyethylene cannulae (0.76 mm ID and 1.22 mm OD, polyethylene tubing, Portex Ltd.; each 6.0 cm in length) filled with normal saline (0.9%, w/v, sodium chloride, Merck) were introduced into these vessels and secured tightly by tying with cotton thread (No. 40, Madura Coats Ltd., India). Furthermore, trachea of one of these rats was exposed and a piece of thread was put loosely around it (recipient). Then the arterial cannula of one of the rats was connected to the venous cannula of another rat and vice versa, using 2.5 cm long piece of damaged thoracic aorta, isolated from carbon tetrachloride treated rats, as mentioned in step 1, Example 2. FIG. 1 schematically illustrates these rats.

In another experiment (used as control experiment) the arterial cannula of one of the rats was connected to the venous cannula of another rat and vice versa, using 2.5 cm long piece of undamaged thoracic aorta, isolated from rats.

The experiment was initiated by opening the clamps on all the vessels, which initiated cross circulation of blood between the two animals. Then the trachea of the recipient was completely blocked by inserting a piece of polyethylene tubing (1.7 mm ID and 2.7 mm OD; Portex Ltd, one end of which was heat-sealed) into it, and subsequently securing it in position with cotton thread. FIG. 2 schematically illustrates these rats.

In spite of the blocked trachea, the recipient survived, as long as the oxygenated blood supply from the donor continued. Survival was monitored by the animal's effort to respire. During the process of cross circulation, when blood passed through the cannula, the platelets came in contact with the internal surface of the damaged vessel and thus were activated. This process led to thrombogenesis, blocked the cannulae, thereby cut off the supply of the oxygenated blood to the recipient, and led to its death.

### Step 3: Drug Administration

In another experiment, pairs of rats were orally treated with antithrombotic agent, viz. Aspirin, at a dose of 20 mg/kg.

Aspirin suspension for oral use was prepared in the vehicle, carboxymethyl cellulose (CMC, 0.5% w/v, Sigma), using polyoxyethylenesorbitan monooleate (Tween 80, Sigma) as a wetting agent.

Vehicle/drug suspension was administered to rats orally, in a volume of 1 ml/kg and was administered once daily for three consecutive days. The experiment was performed on the third day, one hour after the administration of the last dose. The vehicle-treated rats were used as controls.

The survival time of the recipients in the abovementioned experiments was recorded. The results are presented in Table 2.

TABLE 2

| | | | | | Survival time | |
|---|---|---|---|---|---|---|
| Group | | | | No. of | (min) | Level of |
| No. | Treatment | Route | Dose | observations* | Mean ± S.E. | significance |
| 1 | CMC (control) | p.o. | 1 ml/kg | 12 | 5.18 ± 0.99 | *** |
| | (cannula with | | | | | compared |
| | damaged | | | | | to Group 2 |
| | thoracic aorta) | | | | | |
| 2 | CMC (control) | p.o. | 1 ml/kg. | 6 | 41.5 ± 8.65 | ** |
| | (cannula with | | | | | compared |
| | undamaged | | | | | to Group 3 |
| | thoracic aorta) | | | | | |
| 3 | CMC (control) | p.o. | 1 ml/kg | 20 | 10.9 ± 0.70 | *** |
| | (cannula with | | | | | compared |
| | latex tubing; | | | | | to Group 1 |
| | Example 1) | | | | | |
| 4 | Aspirin | p.o. | 20 mg/kg | 7 | 33.9 ± 4.58 | *** |
| | (cannula with | | | | | compared |
| | damaged | | | | | to Group 1 |
| | thoracic aorta) | | | | | n.s. |
| | | | | | | compared |
| | | | | | | to Group 5 |
| 5 | Aspirin | p.o. | 20 mg/kg | 11 | 45.3 ± 4.40 | *** |
| | (cannula with | | | | | compared |
| | latex tubing; | | | | | to Group 3 |
| | Exanmple 1) | | | | | |

*= For each observation one pair of animals consisting of one donor and one recipient animal were used
S.E. = standard error;
** = p < 0.01;
*** = p < 0.001;
n.s. = not significant

### Conclusion:

The results obtained from these experiments, when compared with the earlier results [Example 1], clearly indicate that

1. The process of thrombus formation is quicker if the animals are connected to each other through the cannulae using a damaged blood vessel instead of a piece of normal blood vessel or a piece of latex tubing.

2. Significant increase in the survival time of the recipient is observed due to aspirin treatment and can be picked up by using both the types of cannulae viz. constructed either with a damaged blood vessel or with a latex tubing piece, when compared with the results of the appropriate control animals.

Therefore for routine use of the method, cannulae constructed with a piece of latex tubing can be used to study the anti-thrombotic effect of the drugs/compounds.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as adapted, arranged and configured, constructed and arranged, adapted and configured, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A method for evaluating a test compound for antithrombotic activity, thrombolytic activity, or a combination thereof, the method comprising:
providing a donor test animal and a recipient test animal, the donor and recipient test animals each having been pretreated with test compound, the donor test animal being configured to provide oxygenated blood to the recipient test animal through a first thrombus inducing system;
initiating transport of blood between the donor test animal and the recipient test animal through the first thrombus inducing system;
interrupting respiration of the recipient test animal;
determining the length of time that the recipient test animal survives;
wherein a survival time longer than a predetermined threshold time indicates that the test compound has antithrombotic activity, thrombolytic activity, or a combination thereof.

2. The method of claim 1, wherein the donor test animal and the recipient test animal are configured for cross-circulation between the donor test animal and the recipient test animal.

3. The method of claim 2, wherein a cannula in an artery of the donor test animal is in fluid communication with the first thrombus inducing system and the first thrombus inducing system is in fluid communication with the vein of the recipient test animal; and a cannula in an artery of the recipient test animal is in fluid communication with the first thrombus inducing system and the first thrombus inducing system is in fluid communication with the vein of the donor test animal.

4. The method of claim 1, wherein initiating transport of blood comprises opening blocked artery of the donor test animal, opening blocked artery of the recipient test animal, opening blocked vein of the donor test animal, opening blocked vein of the recipient test animal, or combination thereof.

5. The method of claim 1, wherein interrupting respiration comprises blocking the trachea of the recipient animal.

6. The method of claim 1, wherein determining the length of time that the recipient test animal survives comprises determining the elapsed time from interrupting respiration of the recipient test animal to death of the recipient animal.

7. The method of claim 1, wherein the threshold time is the survival time of a control animal that was not pretreated or that was pretreated with a control substance lacking test compound and therapeutic agent.

8. The method of claim 1, further comprising:
providing a donor control animal and a recipient control animal, the donor and recipient control animals each having been pretreated with control substance, the donor control animal being configured to provide oxygenated blood to the recipient control animal through a second thrombus inducing system;
initiating transport of blood from the donor control animal to the recipient control animal through the second thrombus inducing system;
interrupting respiration of the recipient control animal;
determining the length of time that the recipient control animal survives.

9. The method of claim 8, wherein the control substance comprises vehicle.

10. The method of claim 8, wherein the length of time that the recipient control animal survives is the predetermined threshold time.

11. The method of claim 8, wherein the predetermined threshold time equals the length of time that the recipient control animal survives multiplied by a predetermined number greater than 1.

12. The method of claim 1, further comprising:
providing a donor treated animal and a recipient treated animal; the donor and recipient treated animals each having been pretreated with antithrombotic agent, thrombolytic agent, or a combination thereof; the donor treated animal being configured to provide oxygenated blood to the recipient treated animal through a third thrombus inducing system;
initiating transport of blood from the donor treated animal to the recipient treated animal through the third thrombus inducing system;
interrupting respiration of the recipient treated animal;
determining the length of time that the recipient treated animal survives.

13. The method of claim 1, further comprising:
providing a pair of animals;
treating each of the animals with test compound;
designating one of the animals as the donor test animal and the other animal as the recipient test animal;
configuring the donor test animal to provide oxygenated blood to the recipient test animal through the first thrombus inducing system.

14. The method of claim 8, further comprising:
providing a pair of animals;
treating each of the animals with control substance or sham treating the animals;
designating one of the animals as the donor control animal and the other animal as the recipient control animal;
configuring the donor control animal to provide oxygenated blood to the recipient control animal through the second thrombus inducing system.

15. The method of claim 12, further comprising:
providing a pair of animals;
treating each of the animals with antithrombotic agent, thrombolytic agent, or combination thereof;
designating one of the animals as the donor treated animal and the other animal as the recipient treated animal;
configuring the donor treated animal to provide oxygenated blood to the recipient treated animal through the third thrombus inducing system.

16. A method of assessment of efficacy of compounds for evaluating their antithrombotic and/or thrombolytic potential, by establishing cross circulation between two animals pretreated with antithrombotic agent, and determining the prolongation of survival time of trachea blocked animal.

17. A method of claim 16, wherein the assessment of efficacy of compounds for evaluating their antithrombotic and/or thrombolytic potential, step comprises:
(a) in one experiment, connecting the cannula placed in the carotid artery of one anesthetized animal pretreated with vehicle to the cannula placed in the jugular vein of another anesthetized animal pretreated with vehicle and vice-versa by using a piece of latex tubing or damaged thoracic aorta and identifying one as a donor and another as a recipient;
(b) in another experiment, connecting the cannula placed in the carotid artery of one anesthetized animal pretreated with antithrombotic agent to the cannula placed in the jugular vein of another anesthetized animal pretreated with the same antithrombotic agent and vice-versa by using a piece of latex tubing or damaged thoracic aorta and identifying one as a donor and another as a recipient;
(c) establishing cross circulation of blood between the animals by releasing the clamps placed on the vessels; and blocking trachea of recipient in both experiments (a) and (b);
(d) achieving blocking of trachea as referred to in (c) by tying it tightly with cotton thread after inserting a piece of polyethylene tubing into it, one end of which is heat sealed;
(e) and measuring the survival time of the recipient in the experiments (a) and (b), to indicate the antithrombotic/thrombolytic potential.

18. A method of claim 16, wherein the assessment of efficacy of compounds for evaluating their antithrombotic and/or thrombolytic potential, step comprises:
(a) in one experiment, connecting the cannula placed in the carotid artery of one anesthetized animal pretreated with vehicle to the cannula placed in the jugular vein of another anesthetized animal pretreated with vehicle and vice-versa by using a piece of latex tubing or damaged thoracic aorta and identifying one as a donor and another as a recipient;
(b) in another experiment, connecting the cannula placed in the carotid artery of one anesthetized animal pretreated with potential antithrombotic/thrombolytic agent to the cannula placed in the jugular vein of another anesthetized animal pretreated with the same potential antithrombotic/thrombolytic agent and vice-versa by using a piece of latex tubing or damaged thoracic aorta and identifying one as a donor and another as a recipient;
(c) establishing cross circulation of blood between the animals by releasing the clamps placed on the vessels; and blocking trachea of recipient in both experiments (a) and (b);
(d) achieving blocking of trachea as referred to in (c) by tying it tightly with cotton thread after inserting a piece of polyethylene tubing into it, one end of which is heat sealed;
(e) and measuring the survival time of the recipient in the experiments (a) and (b), to indicate the antithrombotic/thrombolytic potential.

19. The method of claim 1, wherein the animal is non-human.

20. The method of claim 19, wherein the non-human animal is rat, guinea-pig, mouse, hamster, or rabbit.

21. The method of claim 20, wherein the said animal is a rat.

22. The method of claim 12, wherein the antithrombotic/thrombolytic agent is acetylsalicylic acid (Aspirin), dipyridamole, clopidogrel bisulphate, or streptokinase.

23. The method of claim 1, wherein the test compound comprises small molecule, herbal extract, microbial extract, drug, antibody, peptide, or secreted protein.
